(19) United States
(12) Patent Application Publication
(10) Pub. No.: US 2018/0360304 A1
(43) Pub. Date: Dec. 20, 2018

(54) OPHTHALMOLOGIC INFORMATION PROCESSING DEVICE AND NON-TRANSITORY COMPUTER-READABLE STORAGE MEDIUM STORING COMPUTER-READABLE INSTRUCTIONS

(71) Applicant: NIDEK CO., LTD., Gamagori-shi (JP)

(72) Inventors: Tetsuya KANO, Kariya-shi (JP); Norimasa SATAKE, Nukata-gun (JP); Hisanari TORII, Gamagori-shi (JP); Ryosuke SHIBA, Gamagori-shi (JP)

(73) Assignee: NIDEK CO., LTD., Gamagori-shi (JP)

(21) Appl. No.: 16/110,745

(22) Filed: Aug. 23, 2018

Related U.S. Application Data
(63) Continuation of application No. PCT/JP2017/008014, filed on Feb. 28, 2017.

(30) Foreign Application Priority Data
Mar. 4, 2016 (JP) 2016-042881

(51) Int. Cl. A61B 3/00; A61B 3/12; A61B 3/10; G06T 7/00

(52) U.S. Cl. CPC A61B 3/0025; A61B 3/12; G06T 2207/30041; G06T 7/0012; A61B 3/102

(57) ABSTRACT

An ophthalmologic information processing device includes a processor and a memory that stores computer-readable instructions. The computer-readable instructions, when executed by the processor, cause the ophthalmologic information processing device to perform processes that include setting a target position on an ocular fundus of a patient's eye, determining a position of one of a ganglion cell corresponding to a photoreceptor cell present at the target position and a photoreceptor cell corresponding to a ganglion cell present at the target position, and obtaining a first analysis result of a retina at the determined position based on one of a second analysis result and a third analysis result. The second analysis result includes an analysis result of the retina at a center point of the determined position and an analysis result of the retina at an auxiliary point. The third analysis result includes an analysis result of the retina in an analysis region.

### CROSS REFERENCE TO RELATED APPLICATION

This application is a continuation application of International Application No. PCT/JP2017/008014, filed Feb. 28, 2017, which claims priority from Japanese Patent Application No. 2016-042881, filed on Mar. 4, 2016. The disclosure of the foregoing application is hereby incorporated by reference in its entirety.

### BACKGROUND

The present disclosure relates to an ophthalmologic information processing device and a non-transitory computer-readable storage medium that stores computer-readable instructions.

Conventionally, various research has been conducted with respect to a relationship between visual field abnormality and retina abnormality. For example, it is disclosed in publicly known literature that when the retina is seen from the front, positions of photoreceptor cells (cones) that change optical information into signals are displaced from positions of ganglion cells that receive the signals from the photoreceptor cells.

### SUMMARY

When associating a visual field and a state of a retina, it is considered desirable to take into account a displacement between positions of photoreceptor cells and ganglion cells (hereinafter referred to as a “positional displacement between cells”). For example, there is a case in which a visual field test result and the state of the retina are compared with each other. In this case, it is considered effective to compare the visual field test result with the state of the retina at the positions of the ganglion cells that receive signals from the photoreceptor cells located at stimulation positions, instead of the state of the retina at the stimulation positions onto which stimulation light is projected to perform the visual field test. However, even when taking into account the displacement between the cells, a method for appropriately indicating the state of the retina relating to the visual field has not been available as far as known art is concerned.

Embodiments of the broad principles derived herein provide an ophthalmologic information processing device capable of appropriately indicating a state of a retina relating to a visual field, and a non-transitory computer-readable storage medium storing computer-readable instructions.

Embodiments provide an ophthalmologic information processing device that includes a processor, and a memory storing computer-readable instructions. The computer-readable instructions, when executed by the processor, cause the ophthalmologic information processing device to perform processes that include setting a target position on an ocular fundus of a patient's eye, determining a position of one of a ganglion cell corresponding to a photoreceptor cell present at the target position and a photoreceptor cell corresponding to a ganglion cell present at the target position, and obtaining a first analysis result of a retina at the determined position based on one of a second analysis result and a third analysis result, the second analysis result including an analysis result of the retina at a center point of the determined position and an analysis result of the retina at an auxiliary point separated from the center point, and the third analysis result including an analysis result of the retina in an analysis region that is a region including the center point.

Embodiments further provide a non-transitory computer-readable storage medium storing computer-readable instructions that, when executed by a processor of an ophthalmologic information processing device, cause the ophthalmologic information processing device to perform processes that include setting a target position on an ocular fundus of a patient's eye, determining a position of one of a ganglion cell corresponding to a photoreceptor cell present at the target position and a photoreceptor cell corresponding to a ganglion cell present at the target position, and obtaining a first analysis result of a retina at the determined position based on one of a second analysis result and a third analysis result, the second analysis result including an analysis result of the retina at a center point of the determined position and an analysis result of the retina at an auxiliary point separated from the center point, and the third analysis result including an analysis result of the retina in an analysis region that is a region including the center point.

### BRIEF DESCRIPTION OF THE DRAWINGS

FIG. 1 is a block diagram illustrating an electrical configuration of an ophthalmologic information processing system 100 including a PC 1;

FIG. 2 is a diagram illustrating an example of a pattern of stimulation positions 31 arranged on an ocular fundus;

FIG. 3 is a diagram illustrating positions 41 of ganglion cells corresponding to the stimulation positions 31 exemplified in FIG. 2;

FIG. 4 is a diagram illustrating an example of a diagnostic chart 51 displayed in a shape corresponding to the stimulation positions 31;

FIG. 5 is a diagram illustrating an example of a diagnostic chart 61 displayed in a shape corresponding to the positions 41 of the ganglion cells;

FIG. 6 is a flowchart of processing performed by the PC 1 of a present embodiment;

FIG. 7 is an explanatory diagram illustrating a relationship between the determined positions 41 of the ganglion cells, and a center point 43 and auxiliary points 44 that are used to calculate a thickness;

FIG. 8 is a diagram illustrating an example of a diagnosis information display image displayed on a monitor 21;

FIG. 9 is a diagram illustrating an example of a diagnosis information display image displayed on the monitor 21;

FIG. 10 is a diagram illustrating an example of a state of a photoreceptor cell, a ganglion cell, and a nerve fiver 80, which are connected with each other;

FIG. 11 is a diagram illustrating an example of a method for outputting an analysis result of a retina based on a target position specified by a user;

FIG. 12 is a diagram illustrating an example of a method for outputting a plurality of analysis results of a retina based on a plurality of target positions specified by the user;

FIG. 13 is a diagram illustrating an example of a relationship between a target region 88 specified by the user and a region 89 corresponding to the target region 88.

### DETAILED DESCRIPTION

An ophthalmologic information processing device exemplified in the present disclosure is provided with a processor that controls operations of the ophthalmologic information processing device. The processor sets a target position on an ocular fundus of a patient's eye. The processor determines a position of one of a ganglion cell corresponding to a photoreceptor cell present in the target position and a photoreceptor cell corresponding to a ganglion cell present in the target position. The processor obtains a first analysis result of a retina at the determined position based on one of a second analysis result and a third analysis result. The second analysis result includes an analysis result of the retina at a center point of the determined position and an analysis result of the retina at an auxiliary point separated from the center point. The third analysis result includes an analysis result of the retina in an analysis region that is a region including the center point.

According to the ophthalmologic information processing device and a non-transitory computer-readable storage medium storing computer-readable instructions exemplified in the present disclosure, a user can appropriately diagnose a state of the patient's eye based on the first analysis result of the retina, which takes into account a displacement between the positions of the photoreceptor cells and the positions of the ganglion cells. Further, compared with a case in which only an analysis result at a single point in the determined position is obtained, a more appropriate value is obtained. Thus, reliability of the diagnosis is improved.

When obtaining the first analysis result of the retina in the determined position, the processor may obtain the analysis result at the center point of the determined position, and the analysis result at the auxiliary point separated from the center point. Further, the processor may obtain the analysis result of the analysis region including the center point of the determined position. In those cases, compared with a case in which only an analysis result at a single point in the determined position is obtained, a more appropriate value is obtained. Thus, the reliability of the diagnosis is improved. However, the processor can also obtain the first analysis result of the retina at a single point, as an analysis result of the retina at the position of the determined single ganglion cell or at the position of the single photoreceptor cell.

When obtaining the analysis result of the retina at the center point and the analysis result of the retina at the auxiliary point, the processor may set a distance between the center point and the auxiliary point based on an input instruction. Further, when obtaining the analysis result in the analysis region including the center point, the processor may set a size of the analysis region based on an input instruction. In those cases, a layer thickness is obtained in a mode desired by the user.

The processor may determine the position of the ganglion cell corresponding to the photoreceptor cell present at the target position. The processor may obtain the first analysis result at the determined position of the ganglion cell based on one of the second analysis result and the third analysis result. The second analysis result may include the analysis result of the retina at the center point of the determined position and the analysis result of the retina at the auxiliary point separated from the center point. The third analysis result may include the analysis result of the retina in the analysis region that is the region including the center point. In this case, as a result of the target position being set, the first analysis result of the retina at the position of the ganglion cell corresponding to the photoreceptor cell present at the target position is appropriately obtained.

The processor may set, as the target position, at least one stimulation position, the at least one stimulation position being a position, of the ocular fundus of the patient's eye, onto which stimulation light is projected in a visual field test. In this case, the visual field test result, and the analysis result of the retina at the position at which the ganglion cell to which the signal has been sent in the visual field test is present are appropriately associated with each other. Thus, a relationship between the visual field and the state of the retina is appropriately indicated.

However, a method for setting the target position can also be changed. For example, the processor may input an instruction for specifying at least one position on the ocular fundus and set the specified position as the target position. In other words, the processor may allow the user to specify the target position on a photoreceptor cell layer in which the photoreceptor cells are present, or on a ganglion cell layer in which the ganglion cells are present. In this case, an analysis result of the position to be observed by the user is appropriately obtained while taking into account the positional displacement between the photoreceptor cells and the ganglion cells. The analysis result may be a layer thickness, and the like.

When the instruction by the user is input a plurality of times, the processor may set a plurality of positions specified by the user as target positions. The instruction by the user may be an instruction using a click operation of a mouse, or the like. The processor may determine a plurality of positions of the ganglion cells or the photoreceptor cells respectively corresponding to the plurality of set target positions. The processor may obtain the first analysis results of the retina in the plurality of determined positions. In this case, the first analysis results in the plurality of positions to be observed by the user are appropriately obtained.

Further, the target position to be set may be a region (hereinafter referred to as a target region) instead of a point. The processor may determine one of a region of the ganglion cells corresponding to the photoreceptor cells present in the target region and a region of the photoreceptor cells corresponding to the ganglion cells present in the target region. The processor may obtain an average value of the first analysis results of the retina in the determined region. In this case, the first analysis result of the target region is appropriately obtained while taking into account the positional displacement between the photoreceptor cells and the ganglion cells.

The processor may set one of the distance between the center point and the auxiliary point or the size of the analysis region based on an area of the stimulation light projected toward the ocular fundus in the visual field test. In this case, the analysis result of the retina is obtained in an appropriate mode in accordance with the area of the stimulation light. The analysis result of the retina may be the layer thickness, and the like.

The processor may output respective diagnostic information for at least one divided region, of a plurality of the divided regions included in a specific two-dimensional chart, on the basis of results of a plurality of the visual field tests at a plurality of the stimulation positions and of the first analysis results at a plurality of the positions of the ganglion cells corresponding to the plurality of stimulation positions. In this case, the user can also perform the diagnosis while appropriately ascertaining a state of a region of the retina strongly related to visual acuity, for example.

Content of the diagnostic information to be output can be selected as appropriate. For example, the processor may generate the diagnostic information by integrating the visual field test result and the first analysis result of the retina and output the diagnostic information. Further, the processor may associate the visual field test result with the first analysis result of the retina and output the associated information as the diagnostic information.

The processor may display the two-dimensional chart on a front image of the ocular fundus. In this case, the user can perform the diagnosis based on the two-dimensional chart while appropriately ascertaining the position of the ocular fundus. Further, the processor can also display the two-dimensional chart on an image showing blood vessels of the ocular fundus. The image showing the blood vessels of the ocular fundus may be a motion contrast image, and the like. In this case, it also becomes easy for the user to compare a state of the blood vessels with the visual field test result.

When at least one of the plurality of divided regions included in the two-dimensional chart is selected by the user, the processor may notify the user of the at least one stimulation position of the visual field test corresponding to the at least one selected divided region. Further, when at least one of the plurality of stimulation positions of the visual field test is selected by the user, the processor may notify the user of the at least one divided region corresponding to the at least one selected stimulation position. In this case, the user can easily ascertain the relationship between the divided region and the stimulation position.

The processor may display, on a monitor, at least one of a first image, a second image, and a third image along with the two-dimensional chart, the first image showing the stimulation positions, the second image showing information relating to a distribution of a thickness of at least one of layers of the retina, and the third image showing the blood vessels of the retina. The third image showing the blood vessels of the retina may be an OCT motion contrast image, a fluorescence image, or the like. In this case, the user can easily compare at least one of the stimulation positions, the information relating to the retina thickness distribution, and the blood vessels of the retina with the diagnostic information.

The processor may obtain, as the first analysis result of the retina, an analysis result of the thickness of at least one of layers of the retina at the position of the ganglion cell. In this case, the user can appropriately compare the visual field test result with the state of the retina while the displacement between the positions of the photoreceptor cells and the positions of the ganglion cells is taken into account.

Note that the processor may obtain the first analysis result other than the layer thickness as the first analysis result of the retina. For example, the processor may obtain, as the first analysis result of the retina, information relating to at least one of a blood vessel density and a blood vessel area of the retina, which are obtained by analyzing the front image of the ocular fundus, or the motion contrast data of the ocular fundus, or the fluorescence image of the ocular fundus, and the like. In this case, the user can also easily compare the visual field test result at the stimulation position with the state of the blood vessels at the position of the ganglion cell corresponding to the stimulation position.

The processor may determine one of the position of the ganglion cell corresponding to the photoreceptor cell and the position of the photoreceptor cell corresponding to the ganglion cell, based on a model that prescribes relationships between positions of photoreceptor cells and ganglion cells. In this case, a position corresponding to the target position is appropriately determined. Further, the processor may determine the position corresponding to the target position based on a model selected by the user among a plurality of the models. In this case, the position corresponding to the target position is determined by a method desired by the user. Note that the number of the models prepared may be one. Further, the processor may create a model in accordance with an input operation instruction, and determine the position corresponding to the target position based on the created model.

A degree of displacement between the position of the photoreceptor cell and the position of the ganglion cell corresponding to the photoreceptor cell varies depending on sections on the ocular fundus. Thus, as a method for determining one of the position of the ganglion cell corresponding to the photoreceptor cell and the position of the photoreceptor cell corresponding to the ganglion cell, a method for determining the position corresponding to the target position based on a distance between a specific section and the target position on the ocular fundus, and the like may be used. As an example, the specific section on the ocular fundus may be the fovea, and the like. However, when an ocular axial length is not taken into account, it is difficult to accurately calculate the distance between the specific section and the target position on the ocular fundus. As a result, there is a possibility that accuracy of determining the position may deteriorate. Thus, the processor may determine the position corresponding to the target position based on the ocular axial length of the patient's eye. In this case, the accuracy of determining the position is improved.

The processor may display information relating to the first analysis result of the retina as additional information to the visual field test result. In this case, the user can perform the diagnosis by easily comparing the visual field test result and the information relating to the state of the retina. Note that the information relating to the first analysis result of the retina includes not only the first analysis result, but also information obtained as a result of comparing the first analysis result with other data, and the like. The other data may be data of a normal eye, for example.

The processor may associate at least one of the photoreceptor cell and the ganglion cell with a nerve fiber through which a signal from the photoreceptor cell and the ganglion cell passes. In this case, the processor can generate useful information based on a flow of signals generated from the photoreceptor cell. For example, when one of the stimulation positions is selected by the user, the processor may notify the user which one of the nerve fibers corresponds to the photoreceptor cell in the selected stimulation position. In this case, the user can easily compare the visual field test result with the nerve fiber. Further, when one of a plurality of the nerve fibers is selected by the user, the processor may notify the user of the position of the photoreceptor cell corresponding to the selected nerve fiber. Further, the processor may notify the user of a correspondence between the divided regions of the two-dimensional chart and the nerve fibers. As an example, when one of the divided regions of the two-dimensional chart is selected, the processor may notify the user of a region in the vicinity of an optic papilla where the nerve fiber corresponding to the selected divided region is present.

The processor may input an instruction to select which of the first analysis result of the position corresponding to the target position, and the first analysis result of the target position is output. The position corresponding to the target position is one of the position of the ganglion cell corresponding to the photoreceptor cell present at the target position and the position of the photoreceptor cell corresponding to the ganglion cell present at the target position. When an instruction to output the first analysis result of the target position is input, the processor may output the first analysis result of the retina at the target position. In this case, the user can select whether or not to take into account the positional displacement between the photoreceptor cell and the ganglion cell corresponding to the photoreceptor cell, as appropriate. Note that, when the first analysis result of the retina at the target position is output, the first analysis result may be output based on one of the second analysis result and the third analysis result. The second analysis result may include an analysis result of the retina at a center point of the target position and an analysis result of the retina at an auxiliary point separated from the center point. The third analysis result may include an analysis result of the retina in an analysis region including the center point.

When the first analysis result of the target position is obtained based on the analysis result of the retina at each of the center point and the auxiliary point, the processor may obtain the first analysis result at the target position while excluding, among the analysis results of the center point and the auxiliary point, the analysis result of the point whose difference from the analysis results of other points is equal to or greater than a threshold value. Further, when the analysis result of the retina in the analysis region including the center point is obtained, the processor may obtain the first analysis result at the target position while excluding, among the analysis results within the analysis region, the analysis result of the region whose difference from the analysis results of other regions within the analysis region is equal to or greater than a threshold value. In this case, even when a point or a region that generates an abnormal analysis result due to some sort of a fault is included, the first analysis result in the target position is obtained in a more accurate manner.

In an embodiment described below, the ophthalmologic information processing device can perform various operations. However, the ophthalmologic information processing device need not necessarily be capable of performing all of a plurality of operations that will be exemplified in the embodiment described below. For example, the ophthalmologic information processing device may perform the operation of outputting the diagnostic information for each of the divided regions of the two-dimensional chart without performing the operation of obtaining the analysis results of the retina at the center point and the auxiliary point. In this case, the ophthalmologic information processing device can be expressed in the following manner. An ophthalmologic information processing device includes a processor and a memory storing computer-readable instructions. The computer-readable instructions, when executed by the processor, cause the ophthalmologic information processing device to perform processes including: obtaining, of an ocular fundus of a patient's eye, a plurality of stimulation positions that are positions onto which stimulation light is projected in a visual field test; determining a plurality of positions of ganglion cells respectively corresponding to photoreceptor cells at the plurality of stimulation positions; obtaining an analysis result of a retina for each of the plurality of determined positions of the ganglion cells; and outputting respective diagnostic information for at least one divided region, of a plurality of the divided regions included in a specific two-dimensional chart, on the basis of results of a plurality of the visual field tests at each of the plurality of stimulation positions and of the analysis results at the plurality of positions of the ganglion cells corresponding to each of the plurality of stimulation positions.

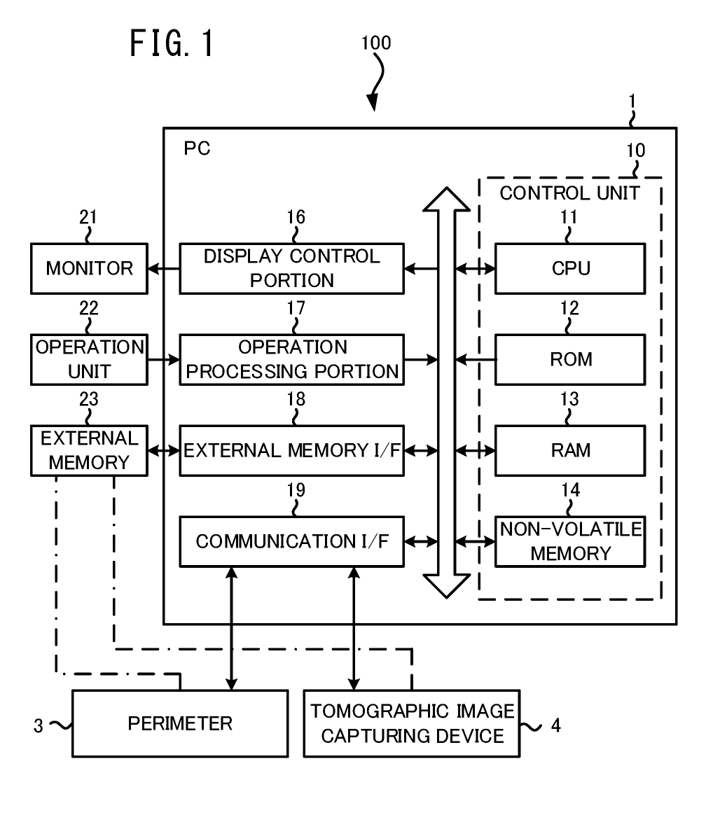

An example of a typical embodiment of the present disclosure will be described below with reference to the appended drawings. First, with reference to FIG. 1, a schematic configuration of an ophthalmologic information processing system 100 of a present embodiment will be described.

As an example, the ophthalmologic information processing system 100 of the present embodiment is provided with a personal computer (hereinafter referred to as a “PC”) 1, a perimeter 3, and a tomographic image capturing device 4. The PC 1 obtains the stimulation positions, and the like in a visual field test performed by the perimeter 3. Further, the PC 1 obtains analysis results of the retina in positions of ganglion cells corresponding to the stimulation positions, based on ocular fundus data generated by the tomographic image capturing device 4. The analysis result of the retina may be the layer thickness of the retina, and the like, for example. In other words, in the present embodiment, the PC 1, which is a separate device from the perimeter 3 and the tomographic image capturing device 4, operates as an ophthalmologic information processing device. However, a device that can operate as the ophthalmologic information processing device is not limited to the PC 1. For example, after obtaining the stimulation positions, and the like from the perimeter 3, the tomographic image capturing device 4 may obtain the analysis results of the retina. The perimeter 3 may also operate as the ophthalmologic information processing device. The visual field test, the tomographic image capturing, an output of diagnostic information, and the like may be all performed by one device.

### PC

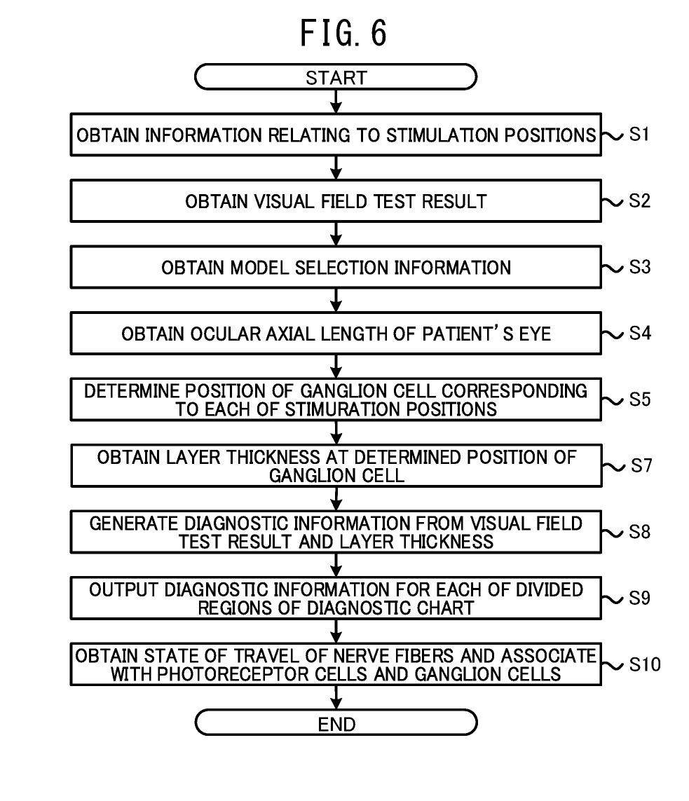

The PC 1 is provided with a control unit 10 that controls operations of the PC 1. The control unit 10 is provided with a CPU 11, a ROM 12, a RAM 13, and a non-volatile memory (NVM) 14. The CPU 11 performs various controls of the PC 1. The ROM 12 stores various programs, default values, and the like. The RAM 13 temporarily stores various types of information. The non-volatile memory 14 is a non-transitory storage medium that can retain stored content even when the power supply is cut off. For example, a hard disk drive, a flash ROM, a removable USB memory, and the like may be used as the non-volatile memory 14. In the present embodiment, an ophthalmologic information processing program for performing processing that will be described later (see FIG. 6), and the like are stored in the non-volatile memory 14.

The control unit 10 is connected to a display control portion 16, an operation processing portion 17, an external memory I/F 18, and a communication I/F 19 via a bus. The display control portion 16 controls a display of a monitor 21. The operation processing portion 17 is connected to an operation unit 22 that receives various operation inputs from the user with respect to the PC 1, and detects those inputs. The operation unit 22 may be a keyboard, a mouse, and the like, for example. The monitor 21 and the operation unit 22 may be externally provided, or may be built into the PC 1. The external memory I/F 18 connects an external memory 23 to the PC 1. Various storage media, such as a UBS memory and a CD-ROM, may be used as the external memory 23. The communication I/F 19 connects the PC1 to an external device. The external device may be the perimeter 3 and the tomographic image capturing device 4, for example. Communication by the communication I/F 19 may be wired communication or wireless communication, and may be performed via the Internet, or the like. The PC 1 can obtain the visual field test result, a three-dimensional image data of the ocular fundus, thickness distribution data of the retina generated as a result of analyzing the three-dimensional image, motion contrast data of the ocular fundus, a front image of the ocular fundus, and the like, via the external memory I/F 18 or the communication I/F 19.

### Perimeter

The perimeter 3 is used to perform the visual field test on the patient's eye. In the present embodiment, perimeters of various configurations can be used. As an example, the perimeter 3 projects (irradiates) stimulation light onto the ocular fundus of the patient's eye whose vision is fixated, causes the patient to answer to what degree the patient has recognized the light, and stores the result. The perimeter 3 performs the visual field test on the patient's eye by sequentially projecting the stimulation light onto each of a plurality of stimulation positions on the ocular fundus and storing the result of the patient's answer for each of the plurality of stimulation positions. Further, the perimeter 3 may have a configuration in which the front image of the ocular fundus is captured. An example of the configuration of the perimeter 3 is disclosed in Japanese Laid-Open Patent Publication No. 2005-102946, and the like.

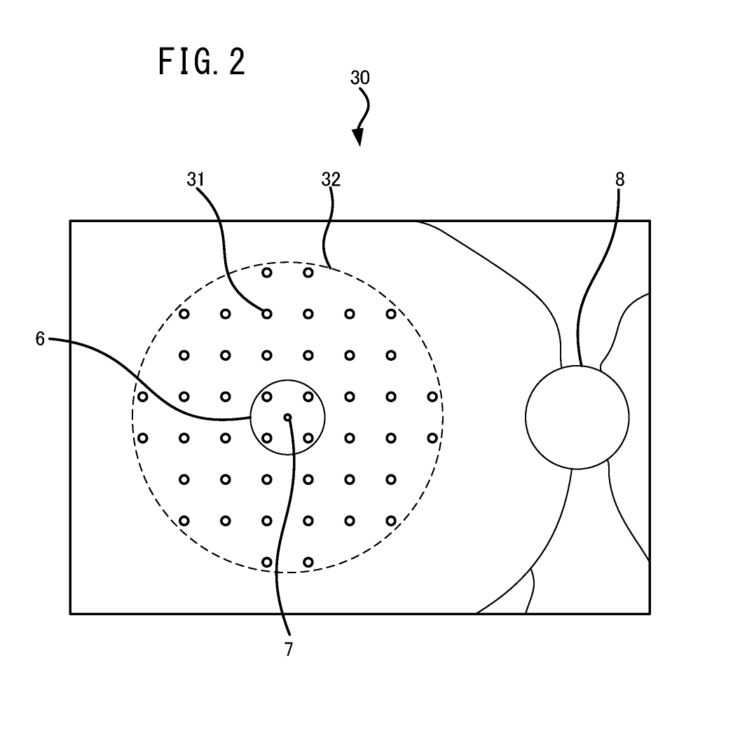

In many cases, the plurality of stimulation positions are arranged in a pattern corresponding to the content of the visual field test, and the like. A stimulation pattern image 30 in FIG. 2 illustrates an example of a pattern of a plurality of stimulation positions 31 arranged on the ocular fundus. In the example illustrated in FIG. 2, a macula 6 and a fovea 7 are positioned on the left side, and an optic papilla 8 is positioned on the right side. In the pattern exemplified in FIG. 2, the plurality of stimulation positions 31 are regularly arranged within a region 32 obtained at a viewing angle of 10 degrees. When the visual field test is performed, the plurality of stimulation positions 31 are projected onto the ocular fundus such that the center of the whole pattern of the plurality of stimulation positions 31 is matched up with the fovea 7. Note that it goes without saying that the pattern of the plurality of stimulation positions 31 is not limited to the example illustrated in FIG. 2.

### Tomographic Image Capturing Device

The tomographic image capturing device 4 can capture at least a tomographic image of the retina of the patient's eye. As an example, in the present embodiment, an OCT is used that captures the tomographic image using light interference technology. The OCT is provided with a light source, a beam splitter, a reference optical system, a scanning unit, and a detector. The light source emits light for capturing the tomographic image. The beam splitter splits the light emitted by the light source into reference light and measurement light. The reference light enters the reference optical system, and the measurement light enters the scanning unit. The reference optical system has a configuration that changes a difference in an optical path length between the measurement light and the reference light. The scanning unit causes the measurement light to perform scanning in two-dimensional directions on tissue. The detector detects an interference state between the measurement light reflected by the tissue, and the reference light that has passed through the reference optical system. The tomographic image capturing device 4 obtains information relating to a depth direction of the tissue by causing the measurement light to perform the scanning and detecting the interference state between the reflected measurement light and the reference light. Based on the obtained information relating to the depth direction, the tomographic image capturing device 4 obtains a tomographic image of an object to be captured. The object to be captured may be the retina, for example. In addition, the tomographic image capturing device 4 can also obtain a three-dimensional image of the retina by causing the measurement light to perform the scanning in the two-dimensional directions on the ocular fundus. Further, the tomographic image capturing device 4 can also obtain data showing a thickness distribution of at least one of the layers of the retina by analyzing the three-dimensional image. The data showing the layer thickness distribution may be the thickness map and the like. Note that processing to obtain the thickness map and the like by analyzing the three-dimensional image may be performed by a device (the PC1 and the like) other than the tomographic image capturing device 4. Further, it goes without saying that a method for obtaining the three-dimensional image can be changed.

In addition, the tomographic image capturing device 4 of the present embodiment can also obtain the front image of the ocular fundus of the patient's eye. The front image of the ocular fundus is a two-dimensional image obtained when seen from the sight direction of the patient's eye. The front image of the ocular fundus can be obtained by various methods. For example, the front image may be obtained by capturing an image of the ocular fundus illuminated by visible light or infrared light. The front image may be obtained by known SLO. A device (a fundus camera and the like) for obtaining the front image of the ocular fundus may be used separately.

As an example, the tomographic image capturing device 4 of the present embodiment can obtain an en-face image as the front image. The en-face image is a front image that is obtained from OCT three-dimensional image data. For example, the en-face image is obtained by integrating the OCT three-dimensional image data in the depth direction. In some cases, a state of travel of the nerve fibers in the retina appears in the en-face image. As will be described in detail later, the PC 1 of the present embodiment can also associate the state of travel of the nerve fibers with at least one of the photoreceptor cell and the ganglion cell.

### Positional Displacement of Corresponding Photoreceptor Cells and Ganglion Cells

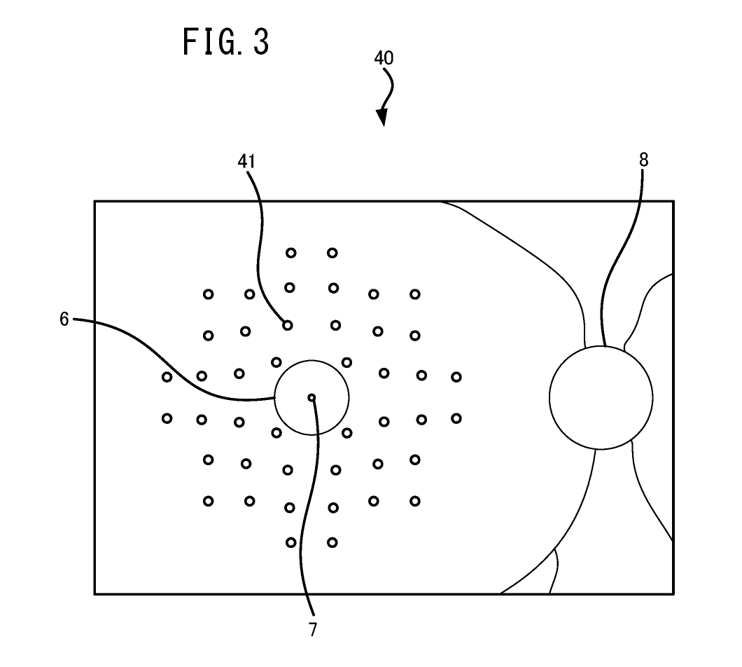

With reference to FIG. 2 and FIG. 3, the positional displacement between the photoreceptor cells and the ganglion cells will be described. In general, the retina includes an inner limiting membrane, a nerve fiber layer (NFL), a ganglion cell layer (GCL), an inner plexiform layer (IPL), an inner granular layer, an outer plexiform layer, a Henle layer, an outer granular layer, an outer limiting membrane, a photoreceptor cell layer, and a retinal pigment epithelium layer of the retina, in that order from a top surface side. A plurality of the photoreceptor cells (cones) are present in the photoreceptor cell layer. Each of the plurality of photoreceptor cells reacts to light, and generates a signal. The signal generated by the photoreceptor cell passes through the Henle layer and so on, moves to the ganglion cell that is present in the ganglion cell layer, and is transmitted to the optic papilla along the extension of the nerve fiber present in the ganglion cell layer. In other words, the signal that is generated by the photoreceptor cell passes through the ganglion cell, the nerve fiber, and the like that are connected to the photoreceptor cell, and is transmitted to the cerebrum. In the present embodiment, the fact that those elements are connected with each other is sometimes described as “corresponding to each other.”

Here, when the ocular fundus is seen from the front, it is known that the positions of the photoreceptor cells and the positions of the ganglion cells are displaced. For example, FIG. 3 is an image 40 that illustrates positions 41 of the ganglion cells corresponding to the stimulation positions 31 exemplified in FIG. 2. When the stimulation light is irradiated on the plurality of stimulation positions 31 using the pattern exemplified in FIG. 2, the positions 41 of the ganglion cells respectively connected to (corresponding to) the plurality of photoreceptor cells in the stimulation positions 31 are displaced from the plurality of the stimulation positions 31, as illustrated in FIG. 3.

“Johann Sjostrand, et al. “Morphometric study of the displacement of retinal ganglion cells subserving cones within the human fovea.” Greafe's Archive for Clinical Experimental Ophthalmology 237.12 (1999): 1014-1023″ will be referred to as Document 1 below. With respect to the positions of the photoreceptor cells and the ganglion cells that are connected with each other, Document 1 describes that a positional displacement x of the photoreceptor cell with respect to the fovea 7 and a positional displacement y of the ganglion cell with respect to the fovea 7 satisfy the following (Expression 1). In addition, in Document 1, an area ratio between the photoreceptor cell and the ganglion cell corresponding to each other is also prescribed.

y=1.29×(x+0.046)^{0.67} (Expression 1)

Below, of the models that prescribe the relationship between the positions of the photoreceptor cells and the positions of the ganglion cells, a model that is described in Document 1 will be referred to as the Sjostrand model. The PC 1 of the present embodiment can determine a position of a ganglion cell connected to a specific photoreceptor cell based on the Sjostrand model.

Further, the PC 1 of the present embodiment can also determine the position of the ganglion cell connected to the photoreceptor cell based on a different model from the Sjostrand model. For example, the relationship between the positions of the photoreceptor cells and the positions of the ganglion cells is prescribed in the following thesis. “Drasdo, Neville, et al. “The length of Henle fibers in the human retina and a model of ganglion receptive field density in the visual field.” Vision Research 47.22 (2007): 2901-2911.” A model prescribed in this thesis is referred to as the Drasdo model. Note that the PC 1 may be capable of determining one of the position of the ganglion cell corresponding to the position of the photoreceptor cell and the position of the photoreceptor cell corresponding to the position of the ganglion cell, based on other models. The position of the ganglion cell corresponding to the position of the photoreceptor cell is also described as the position of the ganglion cell connected to the photoreceptor cell. The position of the photoreceptor cell corresponding to the position of the ganglion cell is also described as the position of the photoreceptor cell connected to the ganglion cell. Further, the PC 1 may create a model in accordance with operations of the operation unit 22 by the user.

Note that a method for determining, based on a model, one of the position of the ganglion cell corresponding to the position of the photoreceptor cell and the position of the photoreceptor cell corresponding to the ganglion cell can be selected as appropriate. For example, in the present embodiment, when the Sjostrand model is used, a program for determining the corresponding position using the above-described (Expression 1) is stored in the non-volatile memory 14. However, the PCI may determine the position by referring to a table and the like that associates the position of the photoreceptor cell with the position of the ganglion cell.

### Diagnostic Chart

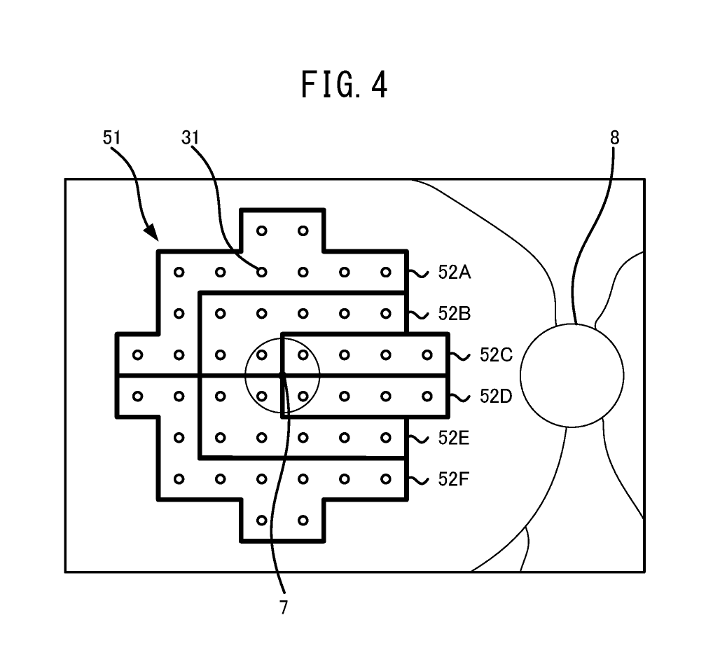

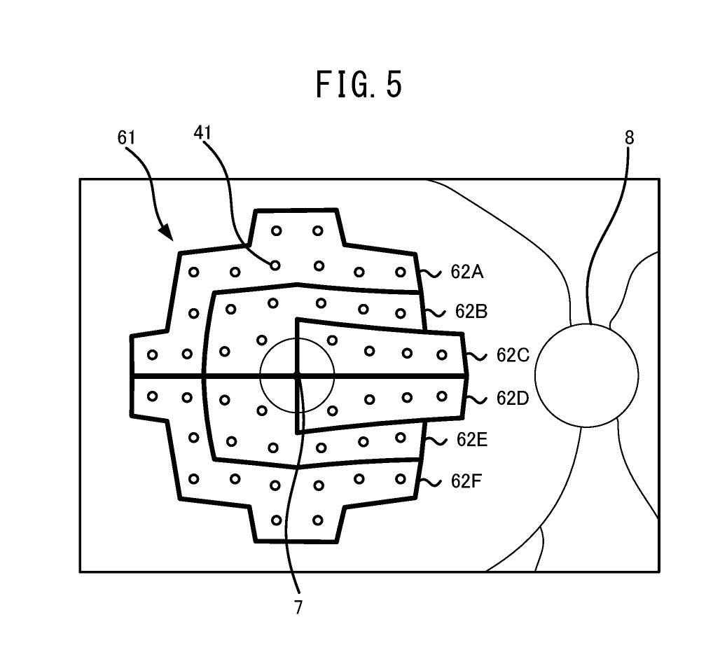

With reference to FIG. 4 and FIG. 5, an example of a diagnostic chart will be described. The diagnostic chart is a two-dimensional chart (a schematic model) on which a plurality of divided regions are arranged. Each of the plurality of divided regions is an output unit of diagnostic information that uses the visual field test result and the analysis result of the retina. The fact that a region strongly relevant to an abnormality of the visual field exists in the retina has been presented in known theses and the like. Thus, by performing diagnosis on the basis of the two-dimensional diagnostic chart, a doctor can appropriately perform the diagnosis of each of the regions of the retina in accordance with a degree of relevancy to the visual field abnormality.

It is sufficient that the diagnostic chart is created appropriately in accordance with the degree of relevancy of each of the regions of the retina to the visual field abnormality. An example of the diagnostic chart is illustrated in FIG. 4. A diagnostic chart 51 exemplified in FIG. 4 includes five divided regions 52A, 52B, 52C, 52D, 52E, and 52F. Each of the divided regions 52 is arranged according to a certain hypothesis, so as to have a different degree of relevancy to the visual field abnormality from the other divided regions 52. For example, the divided region 52C has stronger relevancy to the visual field abnormality than the divided region 52A. If the hypothesis that prescribes the degree of relevancy of each of the regions to the visual field abnormality is different, the shape of the diagnostic chart is also caused to be different. Further, depending on a type of the analysis result used, the diagnostic chart may be changed. The type of the analysis result used includes an analysis result relating to the layer thickness, an analysis result relating to blood vessels, and the like.

The diagnostic chart 51 exemplified in FIG. 4 is displayed using the plurality of stimulation positions 31 as reference points. Thus, the diagnostic chart 51 exemplified in FIG. 4 may be used when the doctor wants to check the diagnostic information using the arrangement of the stimulation positions 31 as the reference, for example. The arrangement of the stimulation positions 31 coincides with the positions of the photoreceptor cells to which stimulation has been applied.

A diagnostic chart 61 exemplified in FIG. 5 is displayed using the plurality of positions 41 of the ganglion cells respectively corresponding to the plurality of stimulation positions 31 (in other words, the positions of the photoreceptor cells) as the reference points. Thus, the diagnostic chart 61 exemplified in FIG. 5 may be used when the doctor wants to check the diagnostic information using the positions 41 of the ganglion cells for which the analysis result of the retina has been obtained as the reference points, for example.

An output of the diagnostic information based on the diagnostic chart is sometimes performed separately for each of the divided regions, is sometimes performed for two or more of the divided regions in an integrated manner, or is sometimes based on the whole diagnostic chart, for example. For example, in the example illustrated in FIG. 4, the case in which two or more of the divided regions 52 are integrated includes a case in which the top half of the divided regions 52A, 52B, and 52C are integrated, a case in which the bottom half of the divided regions 52D, 52E, and 52F are integrated, and the like.

Further, as illustrated in FIG. 4 and FIG. 5, the control unit 10 (the CPU 11) of the PC 1 according to the present embodiment can control the display of the monitor 21 and display the diagnostic chart 51 or 61 on the front image of the ocular fundus. Thus, the user can perform the diagnosis using the diagnostic chart 51 or 61 while appropriately ascertaining the position of the ocular fundus. Note that a method for displaying the diagnostic chart 51 or 61 on the front image can be selected as appropriate. For example, the CPU 11 may display the diagnostic chart 51 or 61 on the front image by distinguishing between a color inside a frame and a color outside the frame of the diagnostic chart 51 or 61, or by distinguishing between a luminance inside the frame and outside the frame, and so on. Further, as illustrated in FIG. 4 and FIG. 5, the CPU 11 may also display the frame of the diagnostic chart 51 or 61 on the front image in a superimposed manner.

### Ophthalmologic Information Control Processing

Processing performed by the CPU 11 of the ophthalmologic information processing device (the PC 1 in the present embodiment) will be described with reference to FIG. 6 and the like. As described above, the ophthalmologic information processing program for performing processing exemplified in FIG. 6 is stored in the non-volatile memory 14. When an instruction to start the output of the diagnostic information is input, the CPU 11 performs processing, which will be described below, in accordance with the ophthalmologic information processing program.

First, the CPU 11 obtains information indicating at least one of the stimulation positions 31 (step S1). As described above, the stimulation positions 31 are positions onto which the stimulation light has been projected in the visual field test. For example, the information indicating the stimulation positions 31 may be coordinate information, or image information on which the stimulation positions 31 are shown. In the present embodiment, the stimulation positions 31 are obtained as target positions.

The CPU 11 obtains the visual field test result for each of the stimulation positions 31 (step S2). As an example, in the present embodiment, the perimeter 3 is used that outputs the visual field test result for each of the stimulation positions 31, while categorizing the results into four stages. Note that the CPU 11 of the present embodiment obtains the information indicating the stimulation positions 31 and the visual field test result from the perimeter 3 via the external memory I/F 18 or the communication I/F 19.

The CPU 11 obtains information relating to an instruction input by the user to select a model from a plurality of models (step S3). As described above, in the present embodiment, the relationship between the positions of the photoreceptor cells and the positions of the ganglion cells is prescribed by a model. Further, in the present embodiment, a plurality of models are prepared. The PC 1 can receive a model selection instruction input by the user via the operation unit 22 and the like.

The CPU 11 obtains an ocular axial length of the patient's eye (step S4). The CPU 11 can obtain the ocular axial length by various methods. For example, the CPU 11 may obtain the ocular axial length of the patient's eye from an ocular axial length measuring device, which measures the ocular axial length using light, ultrasonic waves, or the like, via the external memory 23, a network, and the like. Further, the tomographic image capturing device 4 may measure the ocular axial length using the principle of light interference. In this case, the CPU 11 may obtain the information of the ocular axial length from the tomographic image capturing device 4.

The CPU 11 determines the positions 41 of the ganglion cells corresponding to the target positions (each of the plurality of stimulation positions 31) (step S5). More specifically, the CPU 11 of the present embodiment determines the plurality of positions 41 of the ganglion cells connected to the photoreceptor cells present in each of the plurality of stimulation positions 31, based on a model. Here, the CPU 11 can determine the positions 41 of the ganglion cells based on a model selected by the user from among the plurality of models. Thus, the positions 41 of the ganglion cells are determined by the method desired by the user.

Further, the CPU 11 of the present embodiment can determine the positions 41 of the ganglion cells while taking into account the ocular axial length of the patient's eye. In a case in which an image (the front image, for example) of the ocular fundus is captured, when the ocular axial length of the patient's eye changes, a relationship between a distance between two points on the captured image, and a distance between two actual points on the ocular fundus sometimes changes. For example, when the ocular axial length becomes longer while the field angle of the image capturing is kept constant, a range of the ocular fundus captured becomes wider. In this case, the distance between the two actual points on the ocular fundus looks shorter on the captured image. As in the present embodiment, the model that prescribes the relationship between the positions of the photoreceptor cells and the positions of the ganglion cells sometimes prescribes the positional relationship between the cells based on the distance on the ocular fundus. In this case, the positions 41 of the ganglion cells are not accurately determined, unless the distance on the ocular fundus (a distance between the fovea 7 and each of the points in the present embodiment) is accurately calculated from the image while taking into account the ocular axial length of the patient's eye. The CPU 11 of the present embodiment accurately ascertains the positions of the photoreceptor cells and the ganglion cells on the ocular fundus from the image while taking into account the ocular axial length. As a result, accuracy of determining the positions 41 of the ganglion cells is improved. Note that the above-described method is merely an example. In other words, a specific method for determining the positions 41 of the ganglion cells while taking into account the ocular axial length can be changed as appropriate. Further, it goes without saying that the ocular axial length may also be taken into account when determining the positions of the photoreceptor cells corresponding to the positions of the ganglion cells.

The CPU 11 obtains, as the analysis result of the retina, a thickness of a layer (layer thickness) of the retina in each of the positions 41 of the plurality of ganglion cells determined at step S5 (step S7). A method for obtaining the layer thickness by analyzing the three-dimensional image of the ocular fundus is disclosed in Japanese Laid-Open Patent Publication No. 2010-220771, for example. Note that an example of a layer thickness map, which shows a layer thickness distribution of the retina, is also disclosed in Japanese Laid-Open Patent Publication No. 2010-220771. The CPU 11 of the present embodiment obtains the layer thickness map that is generated in advance by the tomographic image capturing device 4, and obtains the layer thickness at each of the positions 41 of the ganglion cells from the obtained layer thickness map. Note that the method for obtaining the layer thickness can be changed. For example, the layer thickness map may be generated by the PC1 analyzing the three-dimensional image of the ocular fundus. Further, the CPU 11 may obtain only the layer thickness at each of the positions 41 of the ganglion cells by analyzing the three-dimensional image, without generating the layer thickness map that shows the layer thickness distribution at each section of the retina.

The layer of the retina for which the thickness is obtained may be decided as appropriate in accordance with the content of the diagnosis, and the like. As an example, in the present embodiment, the thickness of NFL+GCL+IPL, the thickness of GCL+IPL, the thickness of NFL, and the total thickness of all the layers are each obtained.

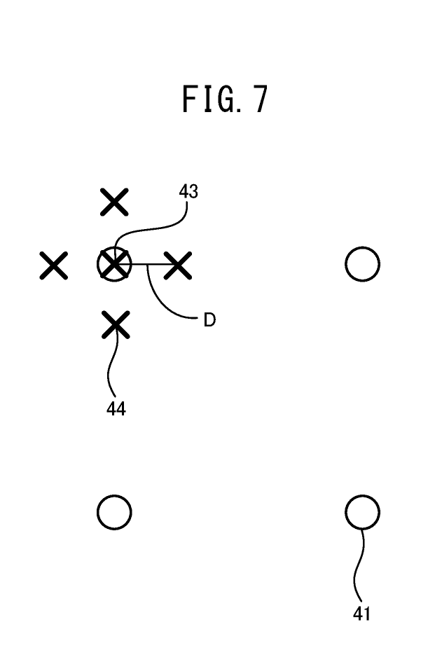

Here, a method for obtaining the layer thickness adopted in the present embodiment will be described in more detail. As illustrated in FIG. 7, the CPU 11 of the present embodiment obtains the layer thickness at one of the determined positions 41 of the ganglion cells based on the layer thicknesses at a plurality of points on the retina. As an example, the CPU 11 of the present embodiment sets a center point 43 at the center of the determined position 41 of the ganglion cell. Further, the CPU 11 sets auxiliary points 44 in positions separated from the center point 43 in directions along the surface of the ocular fundus. The CPU 11 obtains the layer thickness at the position 41 of the ganglion cell based on the layer thickness at each of the set center point 43 and auxiliary points 44.

Note that, in the example illustrated in FIG. 7, the plurality (four in the present embodiment) of the auxiliary points 44 are set at equal distances such that the positions of the plurality of auxiliary points 44 are arranged in a rotationally symmetrical manner around the center point 43. Thus, the layer thickness of an area around the center point 43 is obtained more appropriately. However, a method for setting the auxiliary points 44 can be changed. For example, the number of auxiliary points 44 is not limited to four. Further, the CPU 11 of the present embodiment obtains, as the layer thickness at the position 41 of the ganglion cell, an average value of the layer thickness at each of the center point 43 and the auxiliary points 44. However, this method can be changed. For example, the CPU 11 may cause a weighting of the layer thickness at the center point 43 to be larger than a weighting of the layer thicknesses at the auxiliary points 44.

Further, in the present embodiment, the user can specify a distance D between the center point 43 and the auxiliary point 44. In other words, when an instruction to specify the distance D is input via the operation unit 22 and the like, the CPU 11 sets the auxiliary point 44 using the specified distance D. Thus, the layer thickness is obtained in a mode desired by the user.

Further, the CPU 11 of the present embodiment can set the distance D between the center point 43 and the auxiliary point 44 based on information relating to an area of the stimulation light projected toward the ocular fundus in the visual field test. In this case, the layer thickness is obtained in an appropriate mode in accordance with the projected area of the stimulation light. The information relating to the area of the stimulation light may be obtained from the perimeter 3, or may be input into the PC 1 by the user operating the operation unit 22, for example. Further, in the present embodiment, an area ratio between an area of the positions of the photoreceptor cells and an area of the positions of the corresponding ganglion cells is also prescribed by the model. Thus, the CPU 11 may calculate a region of the ganglion cells corresponding to a region onto which the stimulation light has been projected, using the information relating to the area of the stimulation light and the model that prescribes the area ratio. In this case, the CPU may set the distance D between the center point and the auxiliary point 44 based on the size of the region of the corresponding ganglion cells. However, in this case also, the distance D is set based on the information relating to the area of the stimulation light in the same manner as described above.

A method for calculating the layer thickness at a single point (the single center point 43 or one of the auxiliary points 44, for example) will be described in more detail. A position of a point for which the thickness is calculated (hereinafter referred to as a “thickness calculation point”) is not necessarily aligned with a position of a pixel in the three-dimensional image. On the other hand, when the layer thickness is analyzed from the three-dimensional image, the thickness is calculated using pixels two-dimensionally arranged at equal intervals when seen from the front. Thus, the CPU 11 of the present embodiment calculates the layer thickness of the thickness calculation point using the linear interpolation method, based on the layer thicknesses at four pixels having shortest distances from the thickness calculation point, among the pixels two-dimensionally arranged at equal intervals when the three-dimensional image is seen from the front. Thus, even when the position of the pixel in the three-dimensional image is not matched up with the thickness calculation point, the thickness is calculated more accurately.

Note that the CPU 11 may obtain the layer thickness at the position 41 of the ganglion cell based on the layer thickness in an analysis region including the center point 43. The analysis region may be a circular region, a polygonal region, or the like that expands in directions along the surface of the ocular fundus around the center point 43. The CPU 11 may obtain, as the layer thickness at the position 41 of the ganglion cell, an average value of the layer thicknesses in the analysis region.

The CPU 11 generates the diagnostic information based on the visual field test result and the layer thickness (step S8). As an example, the CPU 11 of the present embodiment generates the diagnostic information by integrating the plurality of visual field test results performed respectively for the plurality of stimulation positions 31, and the layer thicknesses at the positions 41 of the ganglion cells corresponding to the plurality of stimulation positions 31. Various methods can be adopted as a method for integrating the corresponding test results and layer thicknesses. For example, in the present embodiment, the CPU 11 obtains the visual field test result for each of the stimulation positions 31 in four stages (100 points, 40 points, 20 points, and 0 point in this order from the most favorable result). Further, the layer thickness at the position 41 of the ganglion cell corresponding to each of the stimulation positions 31 is categorized into four stages (×1, ×0.75, ×0.5, and ×0.25 in this order from the most favorable result, when compared with the layer thickness of a normal eye) by the CPU 11. The CPU 11 generates the diagnostic information by multiplying the points indicated by the visual field test result by the percentage corresponding to the categorization of the layer thickness.

Note that a method for integrating the visual field test result and the analysis result of the retina (information regarding the layer thickness in the present embodiment) can be changed. Further, the CPU 11 may use, as the diagnostic information, the visual field test result and the analysis result of the retina as they are, without integrating the visual field test result and the analysis result of the retina.

The CPU 11 outputs the diagnostic information for each of the divided regions 52 and 62 of the diagnostic charts 51 and 61 (see FIG. 4 and FIG. 5, for example) (step S9). The CPU 11 of the present embodiment generates the diagnostic information for each of the divided regions 52 and 62 based on one or a plurality of analysis positions (the stimulation positions 31, or the positions 41 of the ganglion cells) included within one of the divided regions 52 or 62. As an example, in the present embodiment, an average of the diagnostic information within the divided region 52 or 62 is generated as the diagnostic information for the divided region 52 or 62 by the CPU 11.

Here, an example of a method for outputting the diagnostic information will be described. There are various methods for outputting the diagnostic information. For example, the CPU 11 may output the diagnostic information by displaying the diagnostic information on the monitor 21. Further, the output of the diagnostic information includes printing, registration of the diagnostic information onto a database, storage of the diagnostic information into a memory, transmission of the diagnostic information via a network, and the like.

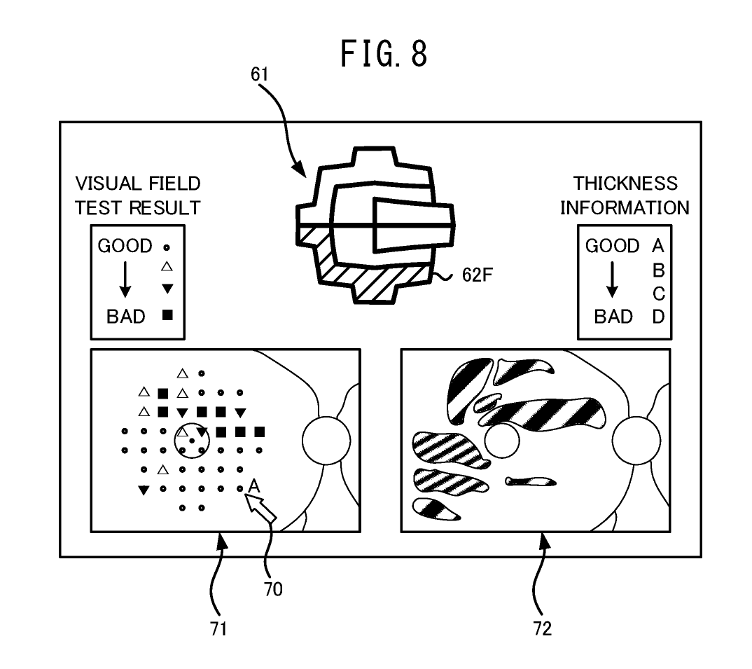

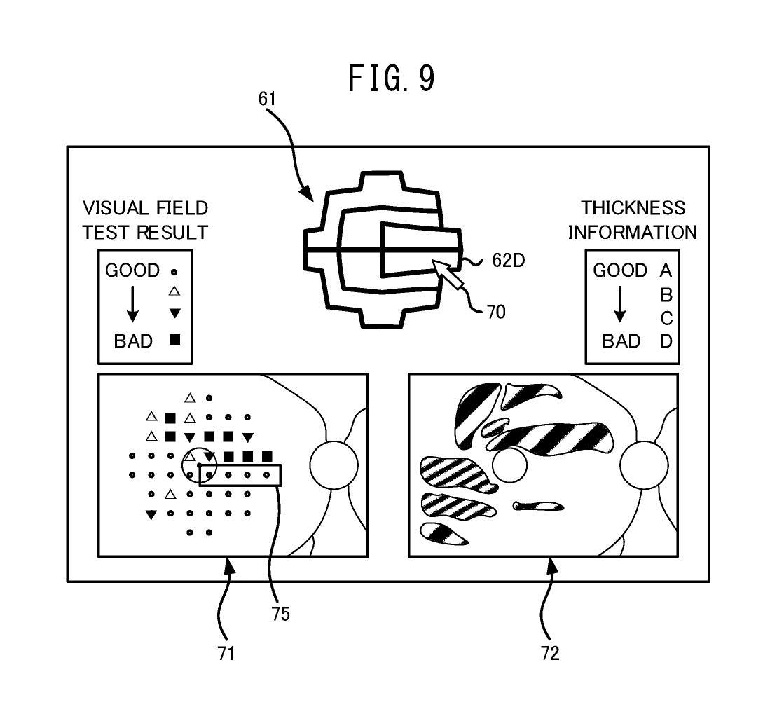

With reference to FIG. 8 and FIG. 9, a method for displaying the diagnostic information in the present embodiment will be described. As illustrated in FIG. 8 and FIG. 9, the CPU 11 of the present embodiment can display at least one of the two types of the diagnostic charts 51 and 61, and can also display the diagnostic information for each of the divided regions 52 and 62 of the diagnostic charts 51 and 61. Although not illustrated in FIG. 8 and FIG. 9, in the present embodiment, the CPU 11 notifies the diagnostic information of the divided region 52 or 62 by changing a color in each of the divided regions 52 and 62. In more detail, the CPU 11 of the present embodiment notifies the user of the diagnostic information by causing the divided regions 52 and 62 having the most favorable analysis results to be displayed in blue, and causing the divided regions 52 and 62 having the least favorable analysis results to be displayed in red. However, a method for notifying the diagnostic information for each of the divided regions 52 and 62 can be changed as appropriate. For example, the CPU 11 may notify the diagnostic information by adding a number or a symbol to each of the divided regions 52 and 62.

Note that, as exemplified in FIG. 4 and FIG. 5, the CPU 11 may display the diagnostic chart 51 or 61 on the front image of the ocular fundus. Further, in FIG. 8 and FIG. 9, only the diagnostic chart 61 is displayed. However, the diagnostic chart 51 (see FIG. 4) and the diagnostic chart 61 may be simultaneously displayed on the monitor 21. Furthermore, different images may be simultaneously displayed on a plurality of the monitors 21.

The CPU 11 of the present embodiment can display on the monitor 21 at least one of a first image showing the stimulation positions 31 in the visual field test and a second image showing the information relating to the layer thickness distribution of the retina, together with the diagnostic charts 51 and 61. Thus, the user can easily compare at least one of the stimulation positions 31 and the information relating to the thickness with the diagnostic information. Further, the CPU 11 may also display a third image showing blood vessels in the retina of the patient's eye, together with the diagnostic chart 51 or 61.

Note that, in examples illustrated in FIG. 8 and FIG. 9, as an example of the first image showing the stimulation positions 31, a visual field test result image 71 is used. In the visual field test result image 71 exemplified in FIG. 8 and FIG. 9, the visual field test result corresponding to each of the stimulation positions 31 is included in addition to the arrangement of the stimulation positions 31 on the ocular fundus. Note that the CPU 11 may also display a result obtained by integrating the visual field test result and the thickness, such that the result corresponds to each of the stimulation positions 31. Further, the CPU 11 can display only the stimulation positions 31 without displaying the visual field test results.

Further, in the examples illustrated in FIG. 8 and FIG. 9, as the second image showing the information relating to the layer thickness distribution of the retina, a layer thickness map 72 is used. In the layer thickness map 72, the layer thickness in each of the sections is indicated on the ocular fundus image by changing the color or the luminance, for example. However, the second image regarding the layer thickness distribution can be changed. For example, by changing the color and the like on the map, for example, the CPU 11 may display results obtained by comparing the layer thickness in each of the sections with the layer thickness of the normal eye.

The CPU 11 of the present embodiment can display the analysis result of the retina as additional information to the visual field test result. As an example, the analysis result of the retina may be the information regarding the layer thickness. In the example illustrated in FIG. 8, the CPU 11 displays the information relating to the layer thickness (“A” that indicates the most favorable result in FIG. 8) as additional information to the visual field test result selected by a cursor 70.

Note that a method for displaying the analysis result of the retina can also be changed. For example, the CPU 11 may add the analysis result of the retina to each of the visual field test results of all of the stimulation positions 31 displayed in the visual field test result image 71. The CPU 11 may also add the analysis result of the retina to each of the plurality of visual field test results respectively corresponding to the specific divided regions 52 and 62. The analysis result of the retina may be displayed by changing the color or the luminance, for example. Further, the information relating to the thickness may be the obtained thickness value itself, or the result obtained based on the comparison with the thickness of the normal eye. Information other than the information regarding the thickness may be displayed as the analysis result of the retina. The information other than the information relating to the thickness may be a blood vessel density, a blood vessel area, and the like, for example.

When the user selects at least one of the plurality of stimulation positions 31, the CPU 11 of the present embodiment can notify the user of the divided region 52 or 62 that includes the selected stimulation position 31, among the divided regions 52 and 62 of the diagnostic charts 51 and 61. In the example illustrated in FIG. 8, in the visual field test result image 71, the stimulation position 31 on the lower right-hand side is selected by the cursor 70. Thus, of the diagnostic chart 61, the CPU 11 notifies the user of the divided region 62F, which includes the selected stimulation position 31. As a result, the user can easily understand the relationship between the divided region 52 or 62 and the stimulation positions 31.

When at least one of the plurality of divided regions 52 and 62 included in the diagnostic charts 51 and 61 is selected, the CPU 11 of the present embodiment can notify the user of the stimulation position 31 corresponding to the selected divided region 52 or 62. In the example illustrated in FIG. 9, in the diagnostic chart 61, the single divided region 62D is selected by the cursor 70. Thus, the CPU 11 notifies the user of the four stimulation positions 31 corresponding to the selected divided region 62D using a frame 75, among the plurality of stimulation positions 31 displayed on the visual field test result image 71. As a result, the user can easily understand the relationship between the divided region 52 or 62 and the stimulation positions 31. Note that a method for the user to select the stimulation positions 31 or the divided region 52 or 62 is not limited to the method of moving the cursor 70. For example, a touch panel, a keyboard, and the like may be used for the selection operation.

Note that, using the diagnostic information, the CPU 11 may suggest to the user a schedule for the next and subsequent visual field tests. For example, in the diagnostic chart 51 or 61, when there exists any of the divided regions 52 and 62 in which an abnormality has been found, the CPU 11 may suggest to the user to perform the next visual field test only for the stimulation positions 31 present inside the divided region 52 or 62 in which the abnormality has been found. The CPU 11 may suggest to the user to obtain a tomographic image including a position for which the visual field result is not good. Further, the CPU 11 may analyze chronological changes in the thickness of the retina, and based on the result, may suggest to the user a schedule for the next visual field test. Furthermore, when there is a section in which the thickness of the retina is gradually becoming thinner, the CPU 11 may suggest to the user to perform the visual field test at least for the stimulation positions 31 corresponding to the section.

The description here returns to FIG. 6. The CPU 11 of the present embodiment obtains information relating to the state of travel of the nerve fibers extending from the ganglion cells to the optic papilla 8. The CPU 11 associates at least one of the photoreceptor cell and the ganglion cell with the nerve fiber connected to the photoreceptor cell and the ganglion cell (step 810). As described above, the signal generated from the photoreceptor cell passes through the ganglion cell and the nerve fiber connected to the photoreceptor cell, and is transmitted to the cerebrum. Thus, by associating the nerve fiber with at least one of the connected photoreceptor cell and ganglion cell, the user can perform an effective diagnosis based on a flow of signals generated from the photoreceptor cell.

Here, an example of a method for obtaining the information relating to the state of travel of the nerve fibers is described. As described above, the tomographic image capturing device 4 of the present embodiment can obtain the en-face image as the front image. In some cases, the state of travel of the nerve fibers in the retina appears in the en-face image. Thus, the CPU 11 may obtain the information relating to the state of travel of the nerve fibers from the en-face image. Further, the state of travel of the nerve fibers of a typical eye may be modeled in advance based on a database of past information and the like. In this case, the CPU 11 may obtain the information of the modeled state of travel. Note that it goes without saying that the method for obtaining the information relating to the state of travel is not limited to these examples. For example, the information relating to the state of travel may be obtained from an OCT motion contrast image, and the like.

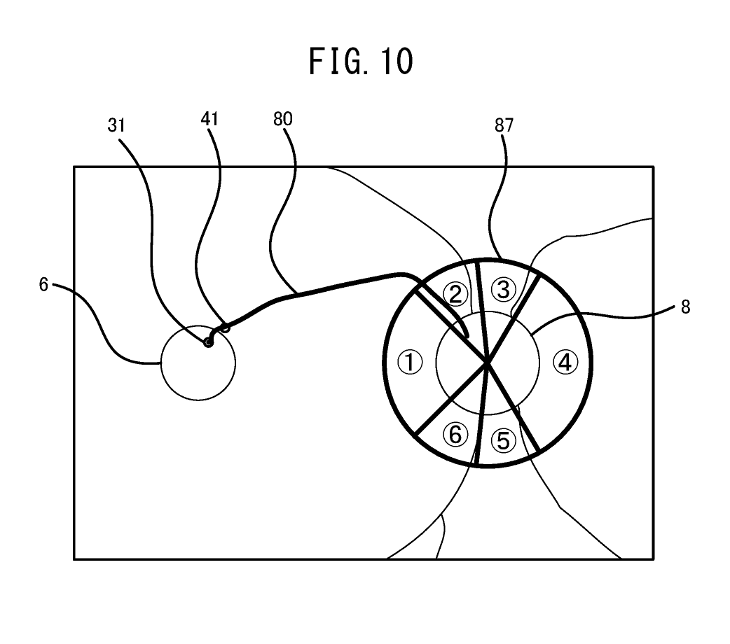

As illustrated in FIG. 10, the CPU 11 can associate with one another the photoreceptor cell, the ganglion cell, and the nerve fiber 80, which are connected with each other, using the state of travel of the nerve fibers. In an example illustrated in FIG. 10, the photoreceptor cell present in the stimulation position 31, the position 41 of the ganglion cell connected to the photoreceptor cell, and the nerve fiber 80 extending from the ganglion cell are illustrated. By performing the association processing at step S10, the CPU 11 can generate various types of useful information. For example, when one of the plurality of stimulation positions 31 is selected by the user, the CPU 11 may notify the user which one of the nerve fibers 80 corresponds to the photoreceptor cell in the stimulation position 31 selected by the user. Further, when one of the plurality of nerve fibers 80 is selected by the user, the CPU 11 may notify the user of the position of the photoreceptor cell connected to the selected nerve fiber 80. Further, when one of the divided regions 52 and 62 of the diagnostic charts 51 and 61 is selected by the user, the CPU 11 may notify the user of the nerve fibers 80 connected to the selected divided region 52 or 62.

Note that the CPU 11 can also associate at least one region in the vicinity of the optic papilla 8 with the photoreceptor cells and the ganglion cells, using the information relating to the nerve fibers 80. In FIG. 10, an example of a peripapillary retinal thickness chart 87 is illustrated. The peripapillary retinal thickness chart 87 is created by dividing the circumference of a circle centered around the optic papilla 8 into a plurality of regions, and is used to perform diagnosis of the layer thickness of the retina in each of the divided regions. Based on the peripapillary retinal thickness chart 87, the user can easily determine the layer thickness of the region that has a significant influence on the visual field, of the periphery of the optic papilla 8. When using the information relating to the nerve fibers 80, the CPU 11 can also notify the user to which of the regions in the peripapillary retinal thickness chart 87 the photoreceptor cell and the ganglion cell correspond.

The technology disclosed in the present embodiment is merely an example. Thus, the technology exemplified in the present embodiment can be changed. For example, in the above-described embodiment, first, the CPU 11 integrates the visual field test result for each of the stimulation positions 31 and the thickness at the position of the corresponding ganglion cell. Then, the CPU 11 generates the diagnostic information of the divided regions 52 and 62 (the average value of the plurality of integrated results, and the like) based on the plurality of integrated results in the divided regions 52 and 62 of the diagnostic charts 51 and 61. However, for example, after calculating an average value of the visual field test results and an average value of the thicknesses for each of the divided regions, the CPU 11 may generate the diagnostic information for the divided regions 52 and 62 by integrating the two calculated average values.

In the above-described embodiment, the CPU 11 obtains the analysis result relating to the layer thickness as the analysis result of the retina at the position of the ganglion cell. However, other analysis results relating to the retina may be obtained as the analysis result of the retina at the position of the ganglion cell. For example, the CPU 11 may obtain an analysis result of the blood vessels at the position of the ganglion cell. The analysis result of the blood vessels may be information relating to the blood vessel density, the area of the blood vessels, and the like. As an example, the analysis result of the blood vessels is obtained from OCT motion contrast data of the ocular fundus. The OCT motion contrast data can be obtained based on a plurality of OCT data of the same position obtained at different timings. Examples of an OCT data calculation method for obtaining the motion contrast data include a method for calculating the intensity difference or amplitude difference in complex OCT data, a method for calculating the variance or standard deviation of the intensity or amplitude of the complex OCT data (Speckle variance), a method for calculating the phase difference or variance of the complex OCT data, a method for calculating the vector differential of the complex OCT data, and a method for multiplying the phase difference of complex OCT signals with the vector differential thereof. Note that, as one of the OCT data calculation methods, for example, one may refer to Japanese Laid-Open Patent Publication No. 2015-131107. Further, the analysis result of the blood vessels may be obtained from front image data based on reflected light from the ocular fundus, front image data based on fluorescence from the ocular fundus, and the like. The analysis data of the blood vessels may be obtained from data obtained by Laser Speckle Flowgraphy (LSFG). Note that an LSFG device is a device that measures the blood flow rate based on speckle signals reflected from blood corpuscles of the eye. Further, an analysis result of the curvature of the ocular fundus may be obtained as the analysis result of the retina. Note that it goes without saying that a plurality of the analysis results of the retina at the position of the ganglion cell may be obtained. The plurality of analysis results of the retina may be the analysis result relating to the layer thickness and the analysis result of the blood vessels, for example.

In the above-described embodiment, the stimulation positions on which the stimulation light is projected in the visual field test are set as the target positions. The analysis results of the retina are obtained at the positions of the ganglion cells corresponding to the photoreceptor cells present in the stimulation positions. However, a method for setting the target positions can be changed. For example, the user may input an instruction to specify the target position in the ophthalmologic information processing device by operating the operation unit, and the like. The CPU 11 may set the position specified by the user as the target position.

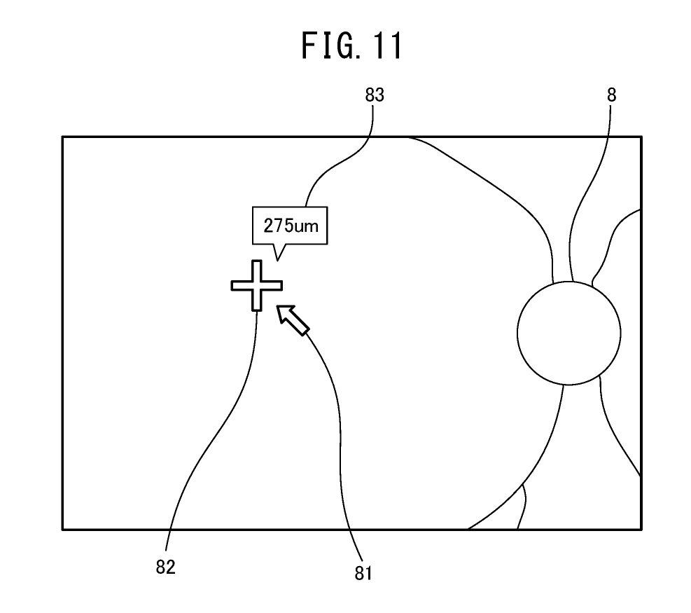

With reference to FIG. 11, an example of a method for outputting the analysis result of the retina based on the target position specified by the user will be described. In an example illustrated in FIG. 11, the user specifies the target position by moving a cursor 81 on the screen using an operation device such a mouse. The CPU 11 sets a tip of the cursor 81 as the target position. Further, the CPU 11 determines the position of the ganglion cell corresponding to the photoreceptor cell in the specified target position, and displays the position of the determined ganglion cell. In the example illustrated in FIG. 11, the CPU 11 displays the position of the ganglion cell corresponding to the target position by displaying, on the screen, a cross-shaped mark 82 that has the position of the determined ganglion cell at the center thereof. The CPU 11 displays the analysis result of the retina at the position of the determined ganglion cell in a frame 83. Therefore, the user can easily ascertain the analysis result at the position of the ganglion cell corresponding to the target position, simply by specifying the target position. Note that, in the example illustrated in FIG. 11, the cursor 81, the mark 82, and the like are displayed on an image of the ocular fundus captured by a fundus photography device (a fundus camera, and the like, for example). However, it goes without saying that the image on which the cursor 81, and the like are displayed can be changed. For example, the cursor 81 and the like may be displayed on the layer thickness map 72 (see FIG. 8). The cursor 81 and the like may also be displayed on the image in which the diagnostic chart 51 or 61 (see FIG. 4 and FIG. 5) is displayed.

Further, the CPU 11 may input an instruction to select which one of the analysis result of the position of the ganglion cell corresponding to the target position or the analysis result of the target position is output. When an instruction to output the analysis result of the position of the ganglion cell corresponding to the target position is input, the CPU outputs the analysis result of the position of the ganglion cell corresponding to the target position, as exemplified in the above-described embodiment. On the other hand, when an instruction to output the analysis result of the target position is input, the CPU 11 outputs the analysis result of the target position. When the analysis result of the target position is output, the CPU 11 may omit the processing (see step S5 in FIG. 6, for example) for determining the positions of the ganglion cells corresponding to the target positions.

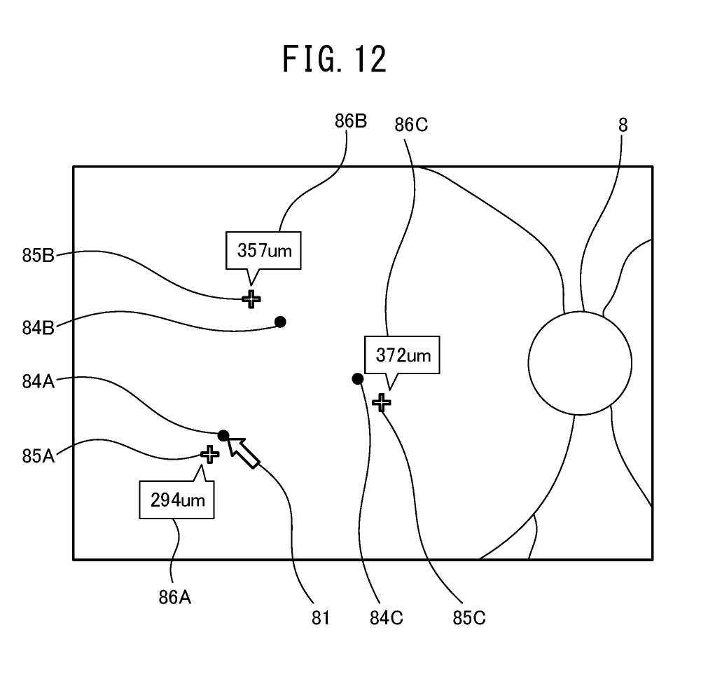

Further, when the instruction to specify the target position is input by the user a plurality of times, the CPU 11 may set a plurality of positions specified by the plurality of times of the input, as the target positions. The CPU 11 may determine the position of the ganglion cell corresponding to each of the plurality of set target positions, and may obtain and output the analysis result of the retina for each of the positions of the plurality of determined ganglion cells. In an example illustrated in FIG. 12, the user specifies the target positions by performing a click operation and the like while moving the cursor 81 on the screen using the operation device such as the mouse. The CPU 11 sets the tip of the cursor 81 obtained at the time when the click operation is performed as the target position. The user can set a plurality of positions as the target positions by performing the click operation a plurality of times. In the example illustrated in FIG. 12, three target positions 84A, 84B, and 84C are set. The CPU 11 determines the position of the ganglion cell corresponding to the photoreceptor cell in each of the specified target positions. The CPU 11 displays the determined positions of the ganglion cells. In the example illustrated in FIG. 12, a mark 85A indicates the position corresponding to the target position 84A. A mark 85B indicates the position corresponding to the target position 84B. A mark 85C indicates the position corresponding to the target position 84C. The CPU 11 displays the analysis result of the retina at each of the determined positions in frames 86A, 86B, and 86C, respectively. Thus, the analysis results for the plurality of positions to be observed by the user are appropriately output.

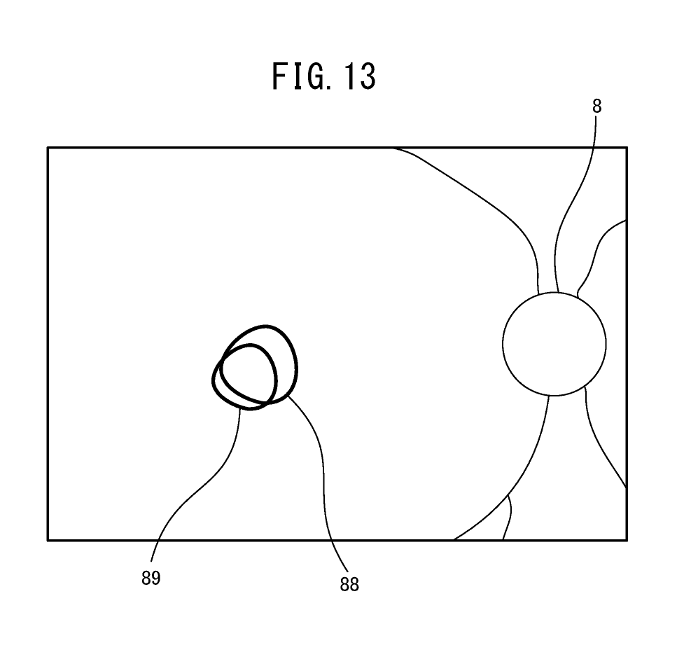

Further, the target position to be set may be a region (hereinafter referred to as a target region) instead of a point. For example, in an example illustrated in FIG. 13, the user specifies a region by operating the operation device such as the mouse. The CPU 11 sets the specified region as a target region 88. Further, the CPU 11 determines a region 89 that includes the positions of the ganglion cells corresponding to the positions of the photoreceptor cells inside the set target region 88. The CPU 11 outputs an average value of the analysis results of the retina in the determined region 89. In this case, an analysis result of the target region 88 is appropriately obtained while taking into account the positional displacement between the photoreceptor cells and the ganglion cells.

Further, in the examples illustrated in FIG. 11 to FIG. 13, when the CPU 11 sets the target position, the CPU 11 determines the position of the ganglion cell corresponding to the photoreceptor cell present at the target position. The CPU 11 obtains the analysis result of the retina at the position of the determined ganglion cell. However, the CPU 11 may determine the position of the photoreceptor cell corresponding to the ganglion cell present at the target position, and may obtain the analysis result of the retina at the position of the determined photoreceptor cell. In this case also, the analysis result of the retina is appropriately obtained while taking into account the positional displacement between the photoreceptor cell and the ganglion cell.

Further, when the analysis result of the target position is obtained based on the analysis result of the retina at each of the center point and the auxiliary points, the CPU 11 may obtain the analysis result of the target point while excluding, of the analysis results of the center point and the auxiliary points, the analysis result of any point whose difference from the analysis results of the other points is equal to or greater than a threshold value. Further, when the analysis result of the retina in the analysis region including the center point is obtained, the CPU 11 may obtain the analysis result at the target position, while excluding, of the analysis results within the analysis region, the analysis result of any region whose difference from the analysis results of the other regions within the analysis region is equal to or greater than a threshold value. Note that, in those cases, the threshold value can be set as appropriate.

The apparatus and methods described above with reference to the various embodiments are merely examples. It goes without saying that they are not confined to the depicted embodiments. While various features have been described in conjunction with the examples outlined above, various alternatives, modifications, variations, and/or improvements of those features and/or examples may be possible. Accordingly, the examples, as set forth above, are intended to be illustrative. Various changes may be made without departing from the broad spirit and scope of the underlying principles.

What is claimed is:

1. An ophthalmologic information processing device comprising:
a processor; and
a memory storing computer-readable instructions, wherein the computer-readable instructions, when executed by the processor, cause the ophthalmologic information processing device to perform processes comprising:
setting a target position on an ocular fundus of a patient's eye;
determining a position of one of a ganglion cell corresponding to a photoreceptor cell present at the target position and a photoreceptor cell corresponding to a ganglion cell present at the target position; and
obtaining a first analysis result of a retina at the determined position based on one of a second analysis result and a third analysis result, the second analysis result including an analysis result of the retina at a center point of the determined position and an analysis result of the retina at an auxiliary point separated from the center point, and the third analysis result including an analysis result of the retina in an analysis region that is a region including the center point.

2. The ophthalmologic information processing device according to claim 1, wherein
the obtaining the first analysis result includes setting one of a distance between the center point and the auxiliary point in the second analysis result and a size of the analysis region in the third analysis result, based on an input instruction.

3. The ophthalmologic information processing device according to claim 1, wherein
the determining the position is determining the position of the ganglion cell corresponding to the photoreceptor cell present at the target position; and
the obtaining the first analysis result includes obtaining the first analysis result of the retina at the determined position of the ganglion cell based on one of the second analysis result and the third analysis result, the second analysis result including the analysis result of the retina at the center point of the determined position of the ganglion cell and the analysis result of the retina at the auxiliary point separated from the center point, and the third analysis result including the analysis result of the retina in the analysis region that is the region including the center point.

4. The ophthalmologic information processing device according to claim 3, wherein
the setting the target position includes setting, as the target position, at least one stimulation position, the at least one stimulation position being a position, of the ocular fundus of the patient's eye, onto which stimulation light is projected in a visual field test.

5. The ophthalmologic information processing device according to claim 4, wherein
the obtaining the first analysis result includes setting one of a distance between the center point and the auxiliary point and a size of the analysis region, based on an area of the stimulation light projected toward the ocular fundus in the visual field test.

6. The ophthalmologic information processing device according to claim 4, wherein
the computer readable instructions, when executed by the processor, further cause the ophthalmologic information processing device to perform a process comprising:
outputting respective diagnostic information for at least one divided region, of a plurality of the divided regions included in a specific two-dimensional chart, on the basis of results of a plurality of the visual field tests at a plurality of the stimulation positions and of the first analysis results at a plurality of the positions of the ganglion cells corresponding to the plurality of stimulation positions.

7. The ophthalmologic information processing device according to claim 6, wherein
the computer readable instructions, when executed by the processor, further cause the ophthalmologic information processing device to perform a process comprising:
controlling a monitor to display the two-dimensional chart on a front image of the ocular fundus.

8. The ophthalmologic information processing device according to claim 6, wherein
the computer readable instructions, when executed by the processor, further cause the ophthalmologic information processing device to perform a process comprising:
performing, when an instruction to select at least one of the plurality of divided regions included in the two-dimensional chart is input, notification of at least one of the stimulation positions corresponding to the at least one selected divided region.

9. The ophthalmologic information processing device according to claim 6, wherein
the computer readable instructions, when executed by the processor, further cause the ophthalmologic information processing device to perform a process comprising:
performing, when an instruction to select at least one of the plurality of stimulation positions is input, notification of the at least one divided region of the two-dimensional chart, the at least one divided region including the at least one selected stimulation position.

10. The ophthalmologic information processing device according to claim 6, wherein
the computer readable instructions, when executed by the processor, further cause the ophthalmologic information processing device to perform a process comprising:
displaying, on the monitor, at least one of a first image, a second image, and a third image along with the two-dimensional chart, the first image showing the stimulation positions in the visual field test, the second image showing information relating to a distribution of a thickness of at least one of layers of the retina, and the third image showing blood vessels of the retina.

11. The ophthalmologic information processing device according to claim 1, wherein
the obtaining the first analysis result includes obtaining an analysis result of a thickness of at least one of layers of the retina at the determined position.

12. The ophthalmologic information processing device according to claim 1, wherein
the determining the position includes
accepting an instruction that is input to select one model from a plurality of models that prescribe relationships between positions of photoreceptor cells and positions of ganglion cells, and
determining one of the position of the ganglion cell corresponding to the photoreceptor cell and the position of the photoreceptor cell corresponding to the ganglion cell, based on the model selected by the instruction.

13. The ophthalmologic information processing device according to claim 1, wherein
the determining the position includes determining one of the position of the ganglion cell corresponding to the photoreceptor cell and the position of the photoreceptor cell corresponding to the ganglion cell, based on an ocular axial length of the patient's eye.

14. The ophthalmologic information processing device according to claim 1, wherein
the computer readable instructions, when executed by the processor, further cause the ophthalmologic information processing device to perform a process comprising:
controlling a monitor to display information relating to the first analysis result of the retina as additional information to a visual field test result.

15. The ophthalmologic information processing device according to claim 1, wherein
the computer readable instructions, when executed by the processor, further cause the ophthalmologic information processing device to perform processes comprising:
obtaining information relating to a state of travel of nerve fibers extending from the ganglion cells to an optic papilla; and
associating at least one of the photoreceptor cell, and the ganglion cell through which a signal generated from the photoreceptor cell passes, with the nerve fiber through which the signal passes.

16. A non-transitory computer-readable storage medium storing computer-readable instructions that, when executed by a processor of an ophthalmologic information processing device, cause the ophthalmologic information processing device to perform processes comprising:
setting a target position on an ocular fundus of a patient's eye;
determining a position of one of a ganglion cell corresponding to a photoreceptor cell present at the target position and a photoreceptor cell corresponding to a ganglion cell present at the target position; and
obtaining a first analysis result of a retina at the determined position based on one of a second analysis result and a third analysis result, the second analysis result including an analysis result of the retina at a center point of the determined position and an analysis result of the retina at an auxiliary point separated from the center point, and the third analysis result including an analysis result of the retina in an analysis region that is a region including the center point.
